(19) United States
(12) Patent Application Publication
(10) Pub. No.: US 2024/0285237 A1
(43) Pub. Date: Aug. 29, 2024

(54) SMART INTRAVENOUS CATHETER SYSTEM

(71) Applicant: B. Braun Melsungen AG, Melsungen (DE)

(72) Inventors: Jo Ann Hang, Penang (MY); Sharienna Sharman, Sungai Buloh (MY); Aik Aun Tan, Palau Penang (MY); Jia Choon Law, Penang (MY)

(73) Assignee: B. Braun Melsungen AG, Melsungen (DE)

(21) Appl. No.: 18/571,019

(22) PCT Filed: Jun. 15, 2022
(86) PCT No.: PCT/EP2022/066375

§ 371 Date: Dec. 15, 2023

Related U.S. Application Data
(60) Provisional application No. 63/211,677, filed on Jun. 17, 2021.

(51) Int. Cl. A61B 5/00; A61B 5/01; A61M 25/06

(52) U.S. Cl. CPC A61B 5/6852; A61B 5/0002; A61B 5/01; A61M 25/0606; A61M 25/0637; A61M 2205/3368; A61M 2205/3553

(57) ABSTRACT

A smart intravenous catheter (IVC) assembly includes a stabilization platform configured to hold an IVC in a fixed position relative to tissue of a patient, and a sensor module mechanically supported by the stabilization platform. The sensor module includes at least one sensor configured to sense a physical characteristic of the tissue of the patient and produce sensed data representing the physical characteristic of tissue of the patient, and a transceiver configured to transmit the sensed data to a smart device remote from the stabilization platform.

### CROSS REFERENCE TO RELATED APPLICATION

This application claims benefit of priority from U.S. Provisional Application No. 63/211,677, filed Jun. 17, 2021. The content of this application is incorporated herein by reference in its entirety and for all purposes.

### FIELD

The subject matter disclosed herein relates to devices, systems and methods for providing a smart intravenous catheter system.

### BACKGROUND

Administering fluids, medications and parenteral nutrition by intravenous (IV) infusion therapy is one of the most common procedures in health care today. Approximately 80% of patients admitted to hospitals receive IV therapy, and around 330 million peripheral IV sets are sold in the United States every year. Simple and effective routine treatment for dehydration, infection and diseases would not be possible without IV therapy. However, even with the advances in this lifesaving procedure over the past years, there is still no simple solution in the market that can continuously monitor and automatically detect if a peripheral IV infusion begins to leak, allowing drugs and fluids designed for IV delivery to escape and accumulate in the subcutaneous tissue. When infiltration occurs, the damage to the patient can range from pain and redness to nerve/tissue damage and limb amputation. The frequency of IV infiltration is alarmingly high. Other complications besides IV infiltrations such as phlebitis and CRBSI (catheter related bloodstream infection) are also problematic. Phlebitis which relates to the inflammation of the vein is the second highly occurred for IV complications. CRBSI which caused by the presence of bacteremia originating from an intravenous catheter (IVC) is one of the most severe complications that may lead infected patients for surgical intervention or even death. Therefore, it is important to detect the IV complications as early as possible before it becomes worst.

### SUMMARY

A smart intravenous catheter (IVC) assembly including a stabilization platform configured to hold an IVC in a fixed position relative to tissue of a patient, and a sensor module mechanically supported by the stabilization platform. The sensor module including at least one sensor configured to sense a physical characteristic of the tissue of the patient and produce sensed data representing the physical characteristic of tissue of the patient, and a transceiver configured to transmit the sensed data to a smart device remote from the stabilization platform.

A smart intravenous catheter (IVC) method including sensing, by a sensor of a sensor module mounted to a stabilization platform holding an IVC in a fixed position relative to tissue of a patient, a physical characteristic of the tissue of the patient. Producing, by the sensor of a sensor module, sensed data representing the physical characteristic of tissue of the patient, transmitting, by a transceiver of the sensor module, the sensed data to a smart device remote from the stabilization platform, and outputting, by the smart device to the patient or to a caregiver, output data related to the sensed data.

A smart intravenous catheter (IVC) assembly comprising a central processor, a sensor module comprising a first temperature sensor configured to measure body temperature at an IVC insertion site on a patient and produce first temperature data, and a first transceiver configured to transmit the first temperature data to the central processor. A second temperature sensor configured to measure body temperature at a reference site on a patient that is remote from the IVC insertion site, and produce second temperature data. A second transceiver configured to transmit the second temperature data to the central processor. The central processor configured to compare the first temperature data to the second temperature data to determine a temperature difference and produce a signal when the temperature difference reaches a predetermined threshold.

### BRIEF DESCRIPTION OF THE FIGURES

FIG. 1A is a cross-sectional view of a section of skin, according to an aspect of the disclosure.

FIG. 1B is a perspective view of an intravenous catheter, according to an aspect of the disclosure.

FIG. 2A is a perspective view of a smart intravenous catheter assembly connected to a reader band, according to an aspect of the disclosure.

FIG. 2B is a perspective view of a sensor module for the smart intravenous catheter assembly in FIG. 2A, according to an aspect of the disclosure.

FIG. 2C shows perspective views of the sensor module in FIG. 2B and an example of light reflection from a section of human skin, according to an aspect of the disclosure.

FIG. 2D is a side view of the smart intravenous catheter assembly in FIG. 2A with the intravenous catheter inserted into the patient, according to an aspect of the disclosure.

FIG. 2E is a perspective view of the smart intravenous catheter assembly in FIG. 2A with a remotely positioned NIR emitter and connected with the sensor module via a cable, according to an aspect of the disclosure.

FIG. 2F is a side view of the smart intravenous catheter assembly in FIG. 2E with the intravenous catheter inserted into the patient, according to an aspect of the disclosure.

FIG. 2G is a cross-sectional view of the smart intravenous catheter assembly in FIG. 2A with a gap for holding the sensor module, according to an aspect of the disclosure.

FIG. 2H is a perspective view of a sensor module with multiple temperature sensors, according to an aspect of the disclosure.

FIG. 3A shows three perspective views of a smart intravenous catheter assembly with stabilization wings, according to an aspect of the disclosure.

FIG. 3B shows a perspective view of a smart intravenous catheter assembly with stabilization wings and an internal sensor module, according to an aspect of the disclosure.

FIG. 4 shows two perspective views of the sensor module in FIG. 3B, according to an aspect of the disclosure.

FIG. 5 is diagram of the smart intravenous catheter system, according to an aspect of the disclosure.

FIG. 6 is a flowchart showing data collection and processing of the smart intravenous catheter system, according to an aspect of the disclosure.

FIG. 7 is a view of a smart device executing a software application of the smart intravenous catheter system, according to an aspect of the disclosure.

FIG. 8 is flowchart showing the operation of the smart intravenous catheter system, according to an aspect of the disclosure.

### DETAILED DESCRIPTION

In the following detailed description, numerous specific details are set forth by way of examples in order to provide a thorough understanding of the relevant teachings. However, it should be apparent to those skilled in the art that the present teachings may be practiced without such details. In other instances, well known methods, procedures, components, and circuitry have been described at a relatively high-level, without detail, in order to avoid unnecessarily obscuring aspects of the present teachings.

### Introduction

The device, system and method described herein provide for detection of intravenous catheter (IVC) complications such as phlebitis, infiltration, CRBSI and others at an early stage. The device, system and method helps clinicians and health care providers (e.g. doctors, nurses, technicians) to diagnose IV complications as early as possible before they get worse. The device, system and method continuously monitors the IVC insertion site and may be integrated with existing patient's electronic medical records (EMR). The device, system and method also helps to secure the catheter and prevent IVC dislodgement or kinking.

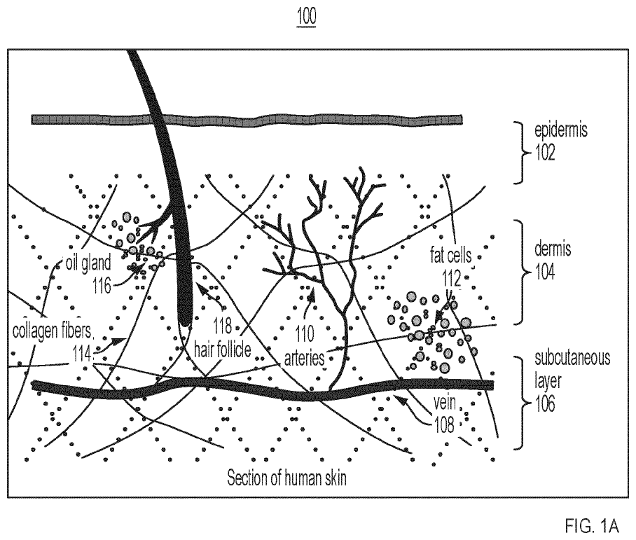

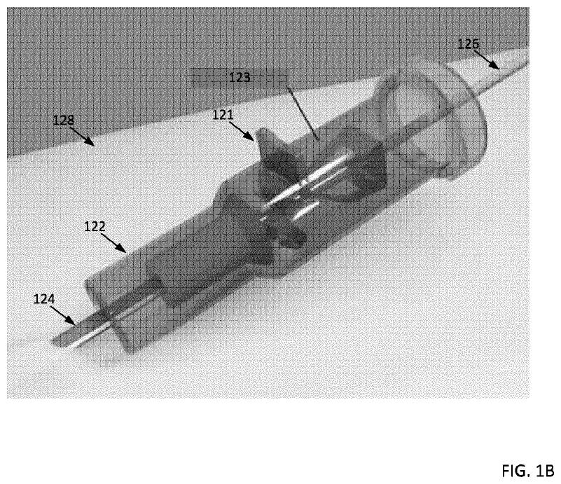

FIG. 1A is a cross-sectional view 100 of a section of human skin including epidermis layer 102, dermis layer 104 and subcutaneous layer 106. When inserting an IVC needle into the human skin, the needle pierces epidermis 102, passes anatomical skin features of the skin layers, such as arteries 110, fat cells 112, collagen fibers 114, oil glands 116 and hair follicles 118 on its way to reaching and piercing a destination vein 108.

An example of an IVC shown in FIG. 1B depicts the IVC inserted into a patient's skin. This insertion could occur at various body parts (e.g. arm, hand, neck, etc.). The IVC may include catheter 124, catheter hub 122, finger push off plate 121, flashback chamber 123 and catheter needle 126. During operation, catheter needle 126 is inserted into the skin and advanced until flashback is visible in flashback chamber 123 confirming that catheter needle 126 has pierced the destination vein. Once flashback is visible, catheter needle 126 is advanced further into the vein and the entire IVC is lowered flat onto the skin of the patient. Using push off plate 121, catheter 124 is advanced off the needle and into the vein until flashback is visible in catheter 124 confirming that catheter 124 is located in the vein. Once catheter 124 is advanced completely into the vein, the needle is withdrawn from catheter hub 122 and a septum (not visible) closes stopping the flow of blood from catheter hub 122. When a luer connection is inserted into catheter hub 122, the septum is forced open allowing blood to be withdrawn or medication to be administered to the patient.

Infiltration, as described above, however, may occur if IV fluid or medications leak into the surrounding tissue. This may be caused by improper placement or dislodgement of the catheter. In one example, in order to avoid and to monitor such a condition, a smart IVC assembly is utilized. The smart IVC assembly includes a stabilization platform (e.g. made from soft material) to reduce dislodgement of the catheter, and a sensor module located between the stabilization platform and the patient's skin. The sensor module may include a temperature sensor and an optical sensor having an emitter and a receiver. The temperature sensor detects the temperature change at the insertion site while the optical sensor emitter uses light, such as near infrared (NIR) light, that is absorbed and reflected by the biological tissue and any fluid present in the biological tissue. The spectrum associated with the NIR has the ability to penetrate tissue and the reflected energy change is captured by the sensor receiver and processed to determine if the region around IVC site is normal or has been infiltrated. In one example, the NIR emitter emits NIR into skin tissue and a photodetector collects the reflected optical signals. As IV fluid infiltrates the interstitial space, the optical density of tissue changes, resulting in a change of the collected optical signals. The presence of infiltrated fluid in subcutaneous layers is then inferred from the differences in the measured signals.

### Device/System Hardware

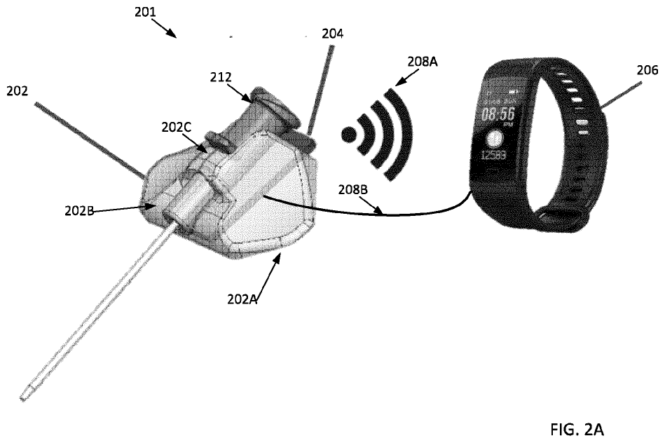

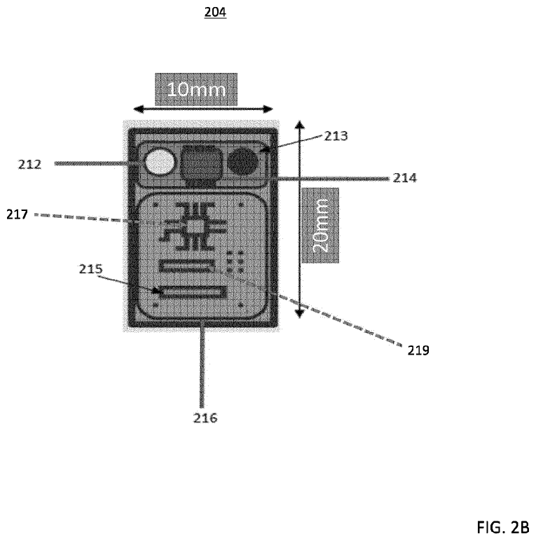

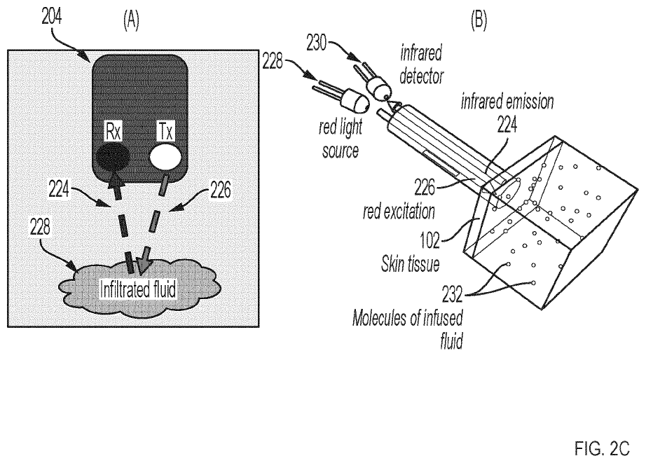

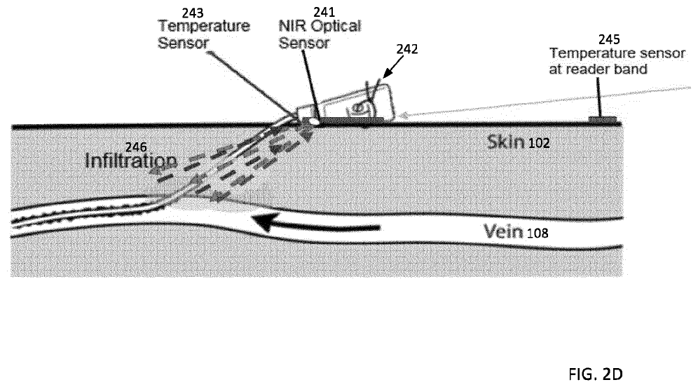

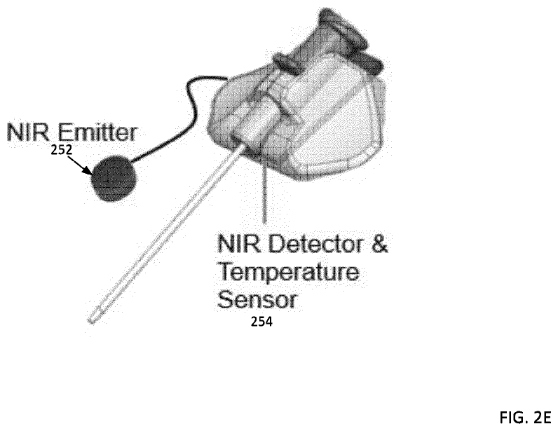

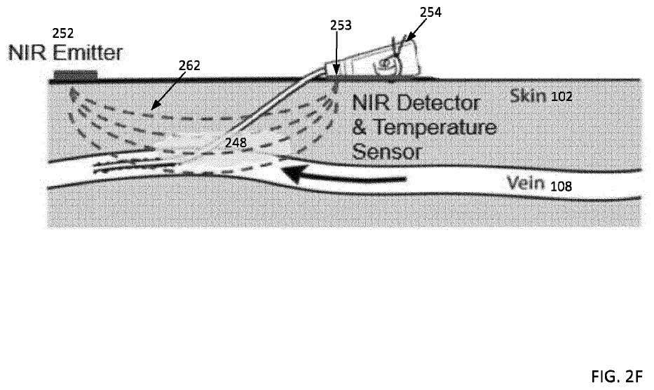

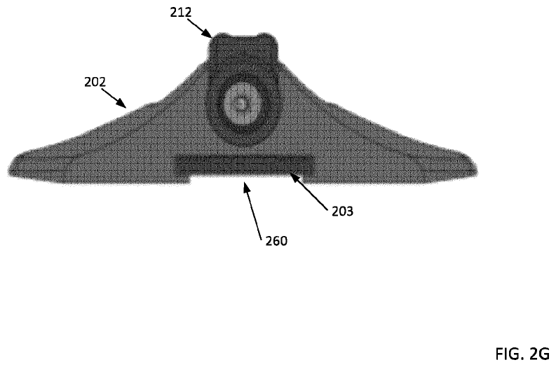

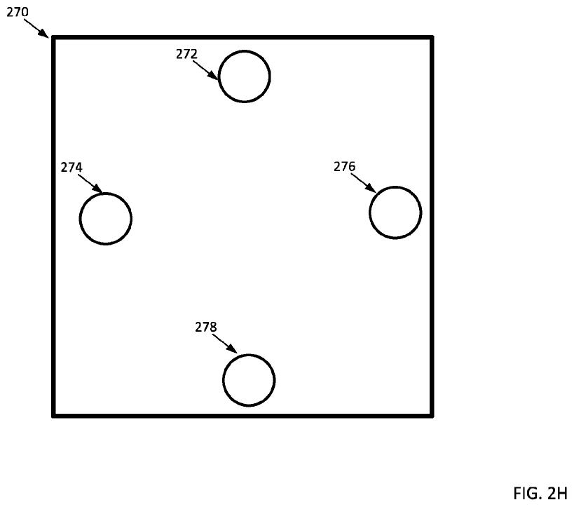

An example of the smart IVC assembly is shown in FIG. 2A where smart IVC catheter assembly 201 is connected (either wired or wirelessly) to a reader band 206. More specifically, smart IVC catheter assembly 201 includes IVC 212, stabilization platform 202 and sensor module 204. In this example, stabilization platform 202 includes base portion 202A, ramp portion 202B, and catheter mount 202C and a slot (not shown) for holding sensor module 204 in place. Stabilization platform 202 may be made of material (e.g. plastic, rubber, etc.) that is sturdy enough to hold IVC 212 in place, while providing comfort and sanitary conditions for the patient. IVC 212 may be any type of catheter capable of being mechanically supported by stabilization platform 202. For example, IVC 212 may be slid or snapped into and secured in catheter mount 202C (e.g. sleeve opening) of stabilization platform 202.

In practice, when a patient is admitted to the hospital they are provided with a reader band 206. During registration, the patient is assigned a patient ID and information regarding their medication and treatment are collected. When the patient is warded, a nurse registers the patient using a mobile application (not shown) through a mobile device (not shown) and the patient's information is then visible in the application. The nurse then performs the IVC procedure on the patient and performs the cannulation process as usual using the catheter stabilization platform having the sensing module inserted therein, and then places dressing on the smart IVC assembly to secure both IVC and the sensor module. In practice, catheter insertion may be performed before or after mounting IVC 212 in stabilization platform 202. Once the catheter is inserted, base portion 202A of stabilization platform 202 rests on the patient's skin (e.g. arm, hand, etc.) and may be affixed with dressing to avoid unwanted movement. Stabilization platform 202 holds IVC 212 at the appropriate angle to avoid catheter kinking and dislodgment.

A connection (e.g. wirelessly through wireless connection 208A, or wired through physical wire 208B) is then established between the sensor module and the reader band. Although not shown, reader band also wirelessly connects with the nurse's mobile device. The wireless connections, such as connection 208A, may be WIFI, Bluetooth, radio frequency identification (RFID) or any other equivalent wireless protocol. Once the connections are made, the nurse begins to wirelessly monitor the injection site for early clinical signs and symptoms through the mobile application on a mobile device. The patient is also able to receive audible and visual notifications directly from the reader band. These notifications may indicate signs and symptoms of complications at the injection site.

Sensor module 204 is shown in more detail in FIG. 2B to include NIR light source 212, NIR light detector 213, temperature sensor 214, a battery 219, supporting electronics 215 and processor 217 that are mounted to printed circuit board 216. It is noted that battery 219 (e.g. lithium-ion) may be a rechargeable battery or a thin battery referred to as a micro-battery (e.g. 0.5 mm×3.6 mm×5.6 mm, 45 mAh) that may be replaceable or may be designed to last the operational life of sensor module 204. Battery 219 is implemented in the sensor module in a configuration where the sensor module is wireless. In a configuration where the sensor module is wired, the power may be supplied directly from the reader band 206 through the cable.

In this example, sensor module 204 has a width of 10 mm and a length of 20 mm. It should be noted that the dimensions of sensor module 204 may vary depending on the dimensions of stabilization platform 202 and the onboard electronics. For example, the dimensions of sensor module 204 may be designed to fit into a mounting slot on the underside of stabilization platform 202. It is also noted that the entire sensor module is hermetically sealed so that it can later be sterilized (e.g. cleaned with alcohol) for reuse with another patient.

During operation, as shown in FIG. 2C, sensor module 204 is used to monitor the presence of infiltrated fluid within the patient's skin. In view (A), for example, sensor module 204 emits light (e.g. NIR light) 226 directed towards the patient's skin located below stabilization platform 202. Light 226 is then absorbed/reflected differently depending on the presence or absence of infiltrated fluid 228. The reflected light 224 is then detected by an NIR receiver (e.g. photodiode) and processed by processor 214 (e.g. to determine if infiltrated fluid is present or not). Another simplified view of this process is shown in view (B) where NIR source 228 emits NIR light 226 which penetrates skin tissue 102 and interacts with the molecules of infused fluid 232. The infrared emission 224 that is reflected by these molecules is detected by NIR detector 230 and then processed by processor 214. Although not shown, the sensor module may also include a biosensor that senses certain molecules in a biological sample of the patient. For example, the biosensor may include a probe that comes into contact with the patient's blood to determine if molecules are present that would indicate infiltration or some other medical issue.

FIG. 2D shows an example 240 of the smart IVC assembly inserted and monitoring the insertion site of a patient. In this example, the catheter of smart IVC assembly 242 is inserted into the patient's vein 108 and the stabilization platform of smart IVC assembly 242 is fixed to the patient's arm. During monitoring, optical sensor 241 of the sensor module, temperature sensor 243 of the sensor module and temperature sensor 245 of the reader band monitor the patient's skin. The sensor module analyzes reflected light from skin 102 to detect the presence or absence of infiltration fluid 246, while the temperature measured by temperature sensor 243 at the insertion site is compared to the patient's body temperature detected by temperature sensor 245 to determine if the injection site temperature exceeds or is less than the body temperature which could indicate complications at the injection site.

For example, if the nurse receives a notification from the mobile application saying there is an average temperature rise (e.g. 2° C.) sensed by the sensor module, then the patient may be diagnosed with phlebitis. If there is a change in the optical properties of the tissue due to fluid leaking, the patient might be diagnosed with infiltration/extravasation. According to one study, a temperature drop of less than 0.7° C./cm is the optimal cut-off for assessing patient with extravasation. As body temperature monitored through mobile application is more than 38.3° C., a critically ill patient should be evaluated for infection and they might be diagnosed with CRBSI.

In another example, smart IVC assembly 250 shown in FIG. 2E physically separates the NIR emitter 252 from the NIR detector 254. For example, NIR detector 254 may be located on the sensor module as described above. However, the NIR emitter 252 is extended via a wire (or wirelessly) so that it may be mounted to a remote location on the user's skin. This may be beneficial to monitor the entire length of the catheter as it is inserted into the patient's skin.

For example, FIG. 2F shows an example of smart IVC assembly 254 inserted and monitoring the insertion site of a patient. In this example, the catheter of smart IVC assembly 254 is inserted into the patient's vein 108 and the stabilization platform of smart IVC assembly 254 is fixed to the patient's skin (e.g. arm). During IVC monitoring, optical emitter 252 is positioned on a remote location of user's skin separate from smart IVC assembly 254 and emits light 262 into the patient's skin. Optical sensor 253 (positioned on the sensor module) analyzes reflected light 262 from skin 102 to detect the presence or absence of infiltration fluid 248. This configuration is beneficial, because it monitors the whole length of the catheter from insertion point to the distal end located in the patient's vein (e.g. the NIR light traverses the length of the catheter). Any infiltration fluid 248 present along this entire area will be detectable due to absorption/reflection of the NIR light due to the fluid.

FIG. 2G shows a cross-sectional view of the smart IVC assembly 201 shown in FIG. 2A. In this view, it is apparent that stabilization platform 202 includes a slot 203 at the bottom surface for inserting and holding sensor module 204 (not shown for clarity). Stabilization platform 202 may be configured to accommodate a non-contact temperature senor by including gap 260 for holding sensor module 204 (when inserted in slot 203) a distance from the user's skin. Alternatively, stabilization platform 202 may be configured to accommodate a contact temperature senor by not including gap 260 such that sensor module 204 (when inserted in slot 203) is held directly against the user's skin. This configuration provides for an accurate sensor reading by ensuring that the sensor is in contact with the user's skin.

Although the sensor module has been described to have one temperature sensor that is compared to a temperature threshold, in other configurations, the sensor module may have multiple temperature sensors to take temperature readings at multiple locations near the insertion site for comparison. Such a configuration may include the main temperature sensor at the insertion site and at least one proximal temperature sensor further from the insertion site. Rather than comparing the main temperature sensor reading to a threshold, the main temperature sensor reading may be compared to the proximal temperature sensor reading to determine the presence of fluid infiltration.

For example, FIG. 2H shows a perspective view of a sensor module 270 having temperature sensor 272 (e.g. main sensor located closest to insertion site) to measure insertion site temperature (IST), and one or more additional temperature sensors 274, 276 and 278 located at various positions proximal to the insertion site to measure a proximal reference temperature (PRT). In this example, PRT may be computed as the average reading of all three of the additional temperature sensors.

In either case, a temperature difference ΔT is computed between IST and PRT (ΔT=IST-PRT). ΔT should be close to zero under normal circumstances. However, if the insertion site begins to heat up due to fluid infiltration, ΔT will be non-zero (e.g. ISR will be greater than PRT). ΔT may therefore be compared to a non-zero threshold to determine if fluid infiltration has occurred on not.

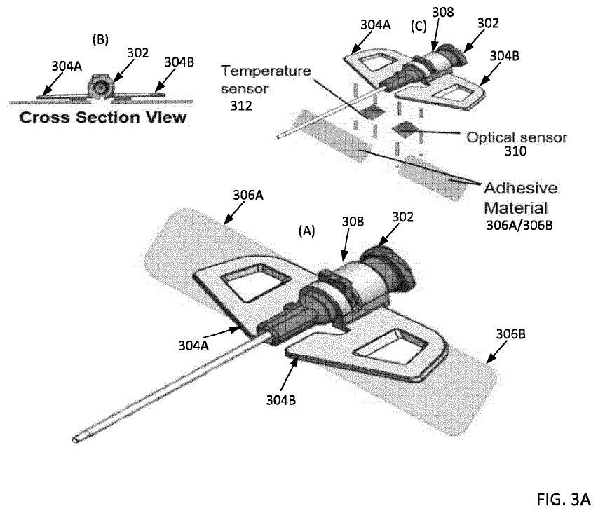

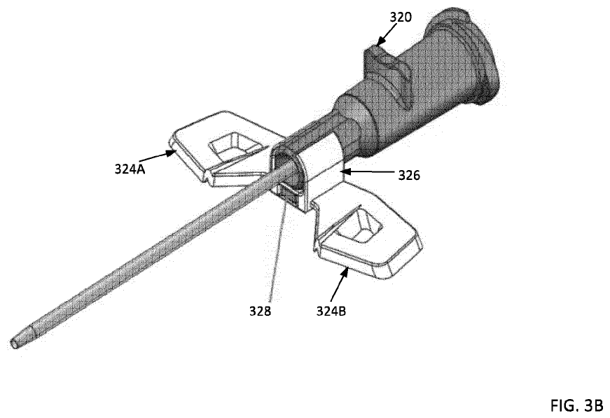

Although FIG. 2A shows smart IVC assembly having stabilization platform 202 with a base portion and a ramp portion, it is noted that the stabilization platform may take different forms. In one example, as shown in FIG. 3A, the stabilization platform may take the form of stabilization wings that hold the IVC in place. For example, as shown in view (A), the stabilization platform may include stabilization wings 304A/304B that may be hinged, flexible or curved to lay flat on the user's skin, and mounting slot 308 that holds IVC 302 in place. Optional adhesive material 306A/306B may also be used to hold stabilization wings 304A/304B in place. As shown in view (C), the stabilization platform includes a sensor module having optical sensor 310 and temperature sensor 312 mounted below stabilization wings 304A/304B. A cross sectional view (B) is also shown for clarity. Optical sensor 310 and temperature sensor 312 operate in a similar manner to those described above with respect to the sensor module in FIG. 2A.

In one example, IVC 302 of the smart IVC assembly in FIG. 3A is inserted into the patient's vein 108 and then stabilization wings 304A/304B are fixed to the patient's skin (e.g. arm). During monitoring, optical sensor 310 of the sensor module, temperature sensor 312 of the sensor module and temperature sensor 243 of the reader band (not shown) monitor the patient's skin. Optical sensor 310 (with integrated emitter or remotely placed emitter) emits/detects light that is then analyzed by a processor (not shown) to determine the presence or absence of infiltration fluid, while the temperature measured by temperature sensor 312 at the insertion site is compared to the patient's body temperature detected by temperature sensor of the band to determine if the injection site temperature exceeds or is less than the body temperature.

Rather than separately placing the optical sensor and temperature sensor below different stabilization wings, the sensors may be integrated into a common sensor module 328 as shown in FIG. 3B. Sensor module 328 may be positioned between stabilization wings 324A/324B as shown in FIG. 3B or under one of the wings (not shown). In addition, rather than completely surrounding IVC 320, mounting slot/bracket 326 may only partially surround and pinch IVC 320 in place.

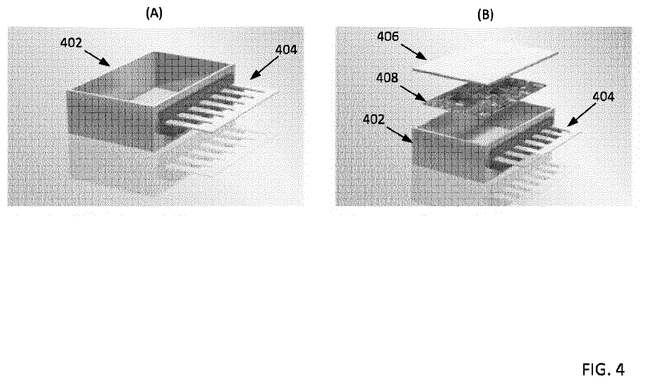

Sensor module 328 may be configured such that it is disposable (e.g. integral with the stabilization platform), or reusable (e.g. may be inserted and extracted from the stabilization platform). For example, a reusable sensor module, as shown in views (A) and (B) of FIG. 4 may include sensor circuit board 408 covered by an autoclave housing having compartment 402 and lid 406. Sensor circuit board 408 may also be electrically connected to pins 404 to provide access for data input and output and/or charging power. The autoclave housing and pins may be hermitically sealed in material (e.g. glass, metal, ceramic, etc.) that can be sterilized with steam and reused. In practice at least a portion of the autoclave housing would be transparent to allow the optical sensor to monitor the patient's skin. This reusable configuration would allow the caregiver to insert the sensor module into an IVC stabilization platform for use on a first patient. Once IVC treatment of the first patient is complete, the caregiver would then remove, sterilize and insert the sensor module into a new IVC stabilization platform for use on a second patient.

In either configuration, the smart IVC assembly provides integrated solution whereby the sensor module and the stabilization platform is integrated together with the IVC. This provides comfort to the patient as bulky device is avoided. The sensor module which is to be inserted at the bottom part of stabilization platform may be reusable and rechargeable (e.g. direct electrical connection charging or inductive charging). In one example, the sensor module is covered (e.g. with transparent plastic casing) which allows the disinfection of it using alcohol and this waterproof coating ensures that the electronic components are impervious to water. In another example, the sensor module is hermetically sealed in material (e.g. glass, metal, ceramic, etc.) that can be sterilized with steam and reused.

### Overall System and Data Processing

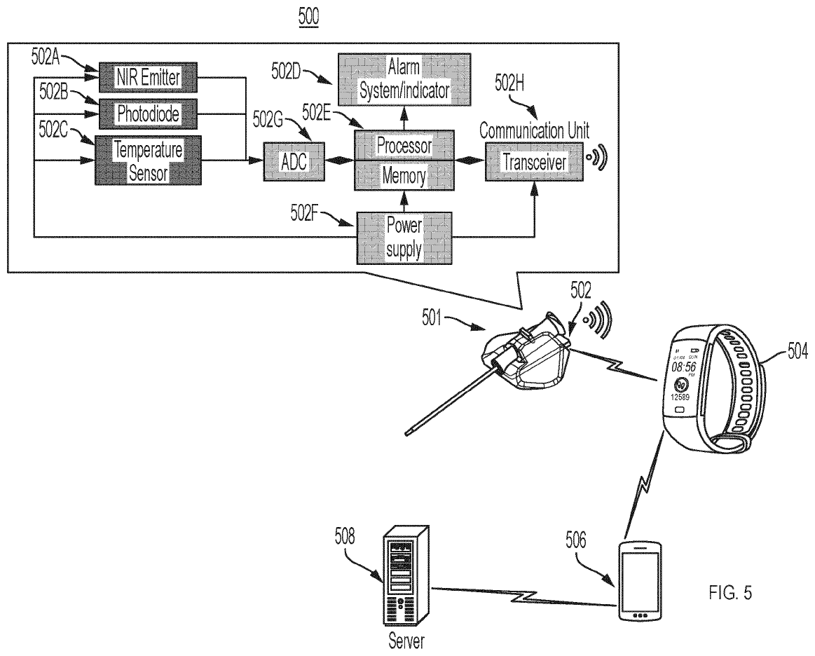

As shown in FIG. 5, in addition to smart IVC assembly 501 and reader band 504, the overall smart IVC system 500 may also include a smart device 506 (e.g. smartphone, tablet, laptop, etc.) and a medical server 508 that work together to monitor the patient's IVC injection site, output alerts and record results. For example, once smart IVC assembly 502 is inserted inside the slot of the stabilization platform and activated (e.g. by a switch or button not shown) and the catheter is inserted into the patient, sensor module 502 begins monitoring the insertion site. Specifically, processor 502E executes a program in the memory to control transceiver 502H to connect wirelessly with reader band 504, control NIR emitter 502A to begin emitting NIR light, control NIR receiver 502B to begin detecting NIR light and control temperature sensor 502C to begin monitoring temperature of the insertion site. Control of NIR emitter 502A, temperature sensor 502C and photodiode 502B are performed through analog to digital converter (ADC) 502G. The various devices/components within sensor module 502 are powered by power supply 502F (e.g. rechargeable battery or a replaceable micro-battery).

The monitored light and temperature data are then sent (e.g. wirelessly) to reader band 504. Specifically, processor 502E may send raw sensor data or processed sensor data to reader band 504. The sensor data may then be forwarded from reader band 504 to smart phone 506. Processing of the raw data may be performed by processor 502E of the sensor module, by a processor (not shown) of the reader band 504, by a processor (not shown) of smart phone 506 or by a combination of all three processors. In either case, the sensor data is processed and the results are displayed to the patient via reader band 504 and to the caregiver via smart device 506. The sensor data may include temperature information and optical properties of the insertion site. In addition, other data and alerts may be computed based on the sensor data and displayed (e.g. alerts of detected infiltration and/or alerts of temperature differences between the temperature at the insertion site and temperature at the reader band).

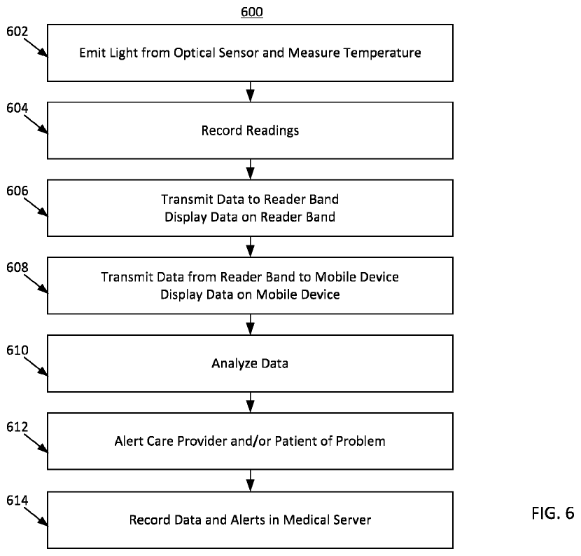

FIG. 6 is a flowchart 600 showing an example of data collection and processing of the smart IVC system. In a first step 602, processor 502E controls the NIR emitter 502A to emit light, controls NIR receiver 502B to receive the emitted light, and controls temperature sensor 502C to monitor temperature of the insertion site. In an optional step 604 processor 502E records (e.g. in a local memory device) data readings representing the received light and the detected temperature. In step 606, processor 502E controls transceiver 502H to transmit the sensor data to reader band 504 which then transmits the sensor data to smart device 506 in step 608. In step 610, reader band 504 and/or smart device 506 analyze the sensor data which is then displayed in step 612 to the patient via reader band 504 and to the caregiver via smart device 506. Among others, the displayed information may include skin quality, skin temperature, alerts and other patient information. In step 614, the sensor data and/or the alerts may then be transmitted from smart device 506 to medical server 508 for storage in patient medical records.

### Software Application and Operational Flow

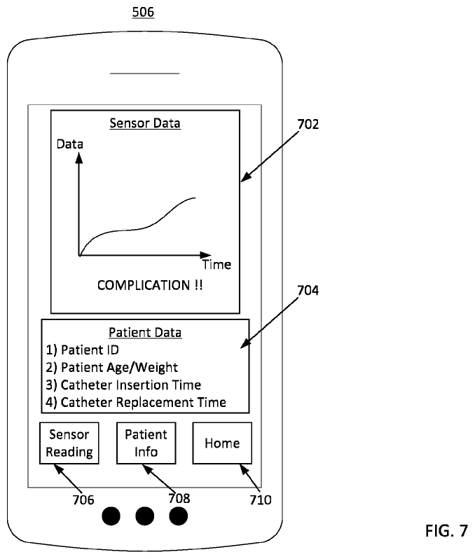

FIG. 7 shows an example of the software application executed on caregiver's smart device 506. In this example, smart device 506 displays sensor data 702, patient data 704 and control buttons 706-710. Sensor data 702 may indicate the quality of the skin at the insertion site as determined by the optical sensor and/or temperature of the skin as determined by the temperature sensor. If this displayed data, for example, exceeds a threshold, then an alert “COMPLICATION” is displayed to alert the caregiver of the situation. For example, in the case of the optical data, the threshold may be: 1) a predetermined threshold of reflected light intensity at specific frequencies that correlate with the presence of infiltration, or 2) a comparison between the reflected light intensity at specific frequencies over time. In the case of the temperature data, the threshold may be: 1) a predetermined threshold of temperature at specific frequencies that correlate with the presence of infiltration, 2) a comparison between the temperature over time, or 3) a comparison of insertion site temperature with body temperature.

Patient data 704 includes, among others, patient ID, patient age/weight, catheter insertion time and catheter replacement time. Control buttons 706-710 may, among others, allow the caregiver to switch between sensor readings (e.g. temperature and optical properties), access/modify patient information, and navigate to the home screen of the application.

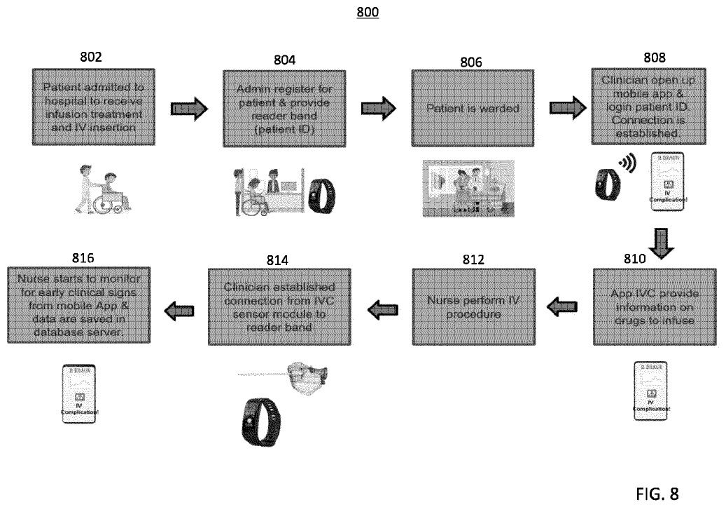

FIG. 8 is flowchart 800 showing the operation of the smart catheter system. In step 802 the patient is admitted to the hospital to receive infusion treatment. In step 804, the patient is registered and provided with a reader band 504 and then warded in step 806. In step 808, the caregiver (nurse tending to the patient) opens the mobile application on smart device 506 and wirelessly connects to reader band 504. In step 810, the mobile application connects to server 508 and sends the patient information (e.g. patient ID) to server 508. Server 508 may then transmit patient medication information to smart device 506. In step 812, the caregiver then inserts smart IVC assembly 501 into the patient. In step 814, the sensor module of smart IVC assembly 501 then connects to reader band 504 and begins monitoring the optical properties and/or temperature of the insertion site. In step 816, the monitored data is then analyzed and displayed to the patient via reader band 504 and to the caregiver via smart device 506. The data as well as any alerts may be forwarded by smart device 506 to medical server 508 for storage in the patient's medical records.

### CONCLUSION

The steps in FIGS. 6-8 may be performed by the sensor module, the reader band, the smart device, the server, or a combination thereof upon loading and executing software code or instructions which are tangibly stored on a tangible computer readable medium, such as on a magnetic medium, e.g., a computer hard drive, an optical medium, e.g., an optical disc, solid-state memory, e.g., flash memory, or other storage media known in the art. In one example, data are encrypted when written to memory, which is beneficial for use in any setting where privacy concerns such as protected health information is concerned. Any of the functionality performed by the computer described herein, such as the steps in FIGS. 6-8 may be implemented in software code or instructions which are tangibly stored on a tangible computer readable medium. Upon loading and executing such software code or instructions by the computer, the controller may perform any of the functionality of the computer described herein, including the steps in FIGS. 6-8 described herein.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” “includes,” “including,” or any other variation thereof, are intended to cover a non-exclusive inclusion, such that a process, method, article, or apparatus that comprises or includes a list of elements or steps does not include only those elements or steps but may include other elements or steps not expressly listed or inherent to such process, method, article, or apparatus. An element preceded by “a” or “an” does not, without further constraints, preclude the existence of additional identical elements in the process, method, article, or apparatus that comprises the element.

Unless otherwise stated, any and all measurements, values, ratings, positions, magnitudes, sizes, and other specifications that are set forth in this specification, including in the claims that follow, are approximate, not exact. Such amounts are intended to have a reasonable range that is consistent with the functions to which they relate and with what is customary in the art to which they pertain. For example, unless expressly stated otherwise, a parameter value or the like may vary by as much as t 10% from the stated amount.

In addition, in the foregoing Detailed Description, it can be seen that various features are grouped together in various examples for the purpose of streamlining the disclosure. This method of disclosure is not to be interpreted as reflecting an intention that the claimed examples require more features than are expressly recited in each claim. Rather, as the following claims reflect, the subject matter to be protected lies in less than all features of any single disclosed example. Thus, the following claims are hereby incorporated into the Detailed Description, with each claim standing on its own as a separately claimed subject matter.

While the foregoing has described what are considered to be the best mode and other examples, it is understood that various modifications may be made therein and that the subject matter disclosed herein may be implemented in various forms and examples, and that they may be applied in numerous applications, only some of which have been described herein. It is intended by the following claims to claim any and all modifications and variations that fall within the true scope of the present concepts.

1. A smart intravenous catheter (IVC) assembly including:
a stabilization platform configured to hold an IVC in a fixed position relative to tissue of a patient; and
a sensor module mechanically supported by the stabilization platform, the sensor module including:
at least one sensor configured to sense a physical characteristic of the tissue of the patient and produce sensed data representing the physical characteristic of tissue of the patient, and
a transceiver configured to transmit the sensed data to a smart device remote from the stabilization platform.

2. The smart intravenous catheter (IVC) assembly of claim 1, wherein the stabilization platform includes a slot, and the sensor module includes a circuit board that is inserted into the slot.

3. The smart intravenous catheter (IVC) assembly according to claim 1,
wherein the at least one sensor includes at least one of a temperature sensor, a NIR emitter, a photodiode, a pressure sensor, a biosensor, or an optical flow sensor for sensing the physical characteristic of the tissue of the patient.

4. The smart intravenous catheter (IVC) assembly according to claim 1, wherein the sensor module is mounted to a portion of the stabilization platform between the IVC and the tissue of the patient to position the at least one sensor a fixed distance from the tissue of the patient.

5. The smart intravenous catheter (IVC) assembly according to claim 1, wherein the at least one sensor is mounted directly on the sensor module or is remotely connected to the sensor module.

6. The smart intravenous catheter (IVC) assembly according to claim 1, wherein sensor module is at least one of coated with an antimicrobial coating, encapsulated in medical grade shrink wrap, or encapsulated in a hermetically sealed housing.

7. The smart intravenous catheter (IVC) assembly according to claim 1,
wherein the stabilization platform includes:
a base portion for stabilizing the stabilization platform against the tissue of the patient, and
a ramp portion for holding the IVC at a fixed position and a fixed angle relative to the tissue of the patient.

8. The smart intravenous catheter (IVC) assembly according to claim 1, wherein the stabilization platform is made of a flexible rubber or a flexible plastic material.

9. The smart intravenous catheter (IVC) assembly according to claim 1, wherein the stabilization platform includes wings that extend laterally to stabilize the stabilization platform against the tissue of the patient.

10. The smart intravenous catheter (IVC) assembly of claim 9, wherein the sensor module is mounted to the wings or to a portion of the stabilization platform between the wings.

11. A m method for detecting IVC complications, the method including:
sensing, by a sensor of a sensor module mounted to a stabilization platform holding an IVC in a fixed position relative to tissue of a patient, a physical characteristic of the tissue of the patient;
producing, by the sensor of a sensor module, sensed data representing the physical characteristic of tissue of the patient;
transmitting, by a transceiver of the sensor module, the sensed data to a smart device remote from the stabilization platform; and
outputting, by the smart device to the patient or to a caregiver, output data related to the sensed data.

12. The method of claim 11, further comprising outputting, by the smart device, at least one of the sensed data or an alert as the output data.

13. The method according to claim 11, further comprising transmitting, by the transceiver of the sensor module, the sensed data to an electronic reader band of the smart device worn by the patient.

14. The method according to claim 11, further comprising transmitting, by the transceiver of the sensor module, the sensed data to the smart device operated by the caregiver.

15. The method according claim 11, further comprising sensing, by the sensor of the sensor module, at least one of a temperature of the tissue, an optical property of the tissue, or a biological sample of the tissue as the physical characteristic.

16. The method according to claim 11, further comprising transmitting, by a software application on the smart device, a control signal to the sensor module, the control signal controlling the sensor module to sense the physical characteristic of the tissue of the patient and transmit the sensed data to the smart device.

17. The method according to claim 11, further comprising transmitting, by the transceiver of the sensor module, the sensed data to a medical database server for storage in a record of the patient.

18. The method according to claim 11, further comprising computing, by the sensor module or the smart device, a health status of the tissue of the patient; and outputting, by the smart device, the health status of the tissue as the output data.

19. The method according to claim 11, further comprising sensing, by the sensor of the sensor module, the physical characteristic of the tissue at a location between the stabilization platform and the tissue.

20. The method according to claim 11, further comprising sensing, by the sensor of the sensor module, the physical characteristic of the tissue at a location remote from the stabilization platform.

21. A smart intravenous catheter (IVC) assembly comprising:
a central processor;
a sensor module comprising:
a first temperature sensor configured to measure body temperature at an IVC insertion site on a patient and produce first temperature data; and
a first transceiver configured to transmit the first temperature data to the central processor;
a second temperature sensor configured to measure body temperature at a reference site on a patient that is remote from the IVC insertion site, and produce second temperature data; and
a second transceiver configured to transmit the second temperature data to the central processor,
the central processor configured to compare the first temperature data to the second temperature data to determine a temperature difference and produce a signal when the temperature difference reaches a predetermined threshold.
